(19) United States
(12) Patent Application Publication
(10) Pub. No.: US 2008/0004482 A1
(43) Pub. Date: Jan. 3, 2008

(54) Radiation source device

(76) Inventors: Eddie Michael Zanrosso, Pasadena, CA (US); Jeffrey James Kempton, Burbank, CA (US)

Correspondence Address: CHRISTIE, PARKER & HALE, LLP, PO BOX 7068, PASADENA, CA, 91109-7068, US

(21) Appl. No.: 11/479,380

(22) Filed: Jun. 30, 2006

(51) Int. Cl. A61N 5/00; A61M 36/00

(52) U.S. Cl. 600/8; 600/3

(57) ABSTRACT

A radiation source device. The radiation source device has a radiolucent window portion, such as formed of beryllium, with a front window and a sleeve portion having an outer surface and a window portion cavity therein. A primary element, e.g., a metal wire, is provided that has a radioactive end, which primary element is received in the window portion cavity with its radioactive end adjacent to the front window and a seal is located between an outer wall of the primary element and an inner wall of the window portion. A radiopaque capsule is provided and has an open end that is sized to receive the sleeve portion of the radiolucent window portion and the primary element. A secondary seal between the capsule and the radiolucent window portion is provided.

### BACKGROUND

The invention relates to radiation sources, namely a small, sealed radiation source that includes a capsule body a radiolucent window, and a radioactive sealed inside of the capsule body behind the window radiolucent.

X-ray radiation sources are used in a wide variety of applications. For example, they can be used to calibrate equipment. X-ray radiation sources are also used in thickness or density measuring devices (e.g., aluminum foil manufacturing, etc.), where the radiation source is placed opposite a detector device with the material the thickness of which is being measured between. A growing market for X-ray radiation sources are in energy-dispersive X-ray fluorescence (EDXRF) analyzers. For example, Thermo Electron Corporation, of Billerica, Mass., and KeyMaster Technology, Inc., of Kennewick, Wash., manufacture a line of EDXRF analyzers. These EDXRF analyzers are able to quickly, non-destructively determine the heavy elemental composition of a variety of materials and items, including metal and precious metal samples, rocks and soil, slurries and liquid samples, painted surfaces, including wood, concrete, plaster, drywall and other building materials, dust collected on wipe samples and airborne heavy elements collected on filters. These EDXRF analyzers function by measuring the characteristic fluorescence x-rays emitted by a sample. Every atomic element present in a sample produces a unique set of characteristic X-rays that is a fingerprint for that specific element. EDXRF analyzers determine the chemistry of a sample by measuring the spectrum of the characteristic x-rays emitted by the different elements in the sample when it is illuminated by high energy photons (X-rays or gamma rays). A fluorescent X-ray is created when a photon of sufficient energy strikes an atom in the sample, dislodging an electron from one of the atom's inner orbital shells (lower quantum energy states). The atom regains stability, filling the vacancy left in the inner orbital shell with an electron from one of the atom's higher quantum energy orbital shells. The electron drops to the lower energy state by releasing a fluorescent X-ray, and the energy of this fluorescent x-ray (typically measured in electron volts, eV) is equal to the specific difference in energy between two quantum states of the dropping electron. The high energy photons (X-rays or gamma rays) are provided by an X-ray or gamma particle source. In order to allow EDXRF analyzers to remain portable, easy to use and affordable, it is important to keep their size and weight down. Accordingly, it is important to have affordable and small X-ray or gamma ray sources available.

Presently, small X-ray sources typically comprise a metal shell (e.g., stainless steel) with an open end into which a holder is inserted. The holder has a front face which carries the radiation source. The radiation source can comprise a radioactive foil or other material. In front of the foil, to seal off the open end of the metal shell is a radiolucent window, such as beryllium, which is brazed in place to seal it off. One large producer of beryllium windows for X-ray sources is Brush Wellman, of Cleveland, Ohio. Unfortunately, brazing small sized beryllium windows is difficult when done on small scale devices.

Accordingly, there is a need for an improved design of X-ray sources and a method of manufacturing same.

### SUMMARY OF THE INVENTION

The radiation source device of the invention comprises a capsule made of a radiopaque material, such as stainless steel. The capsule can have a generally cylindrical shape with a closed end and an open end defining a cavity therein. The open end has a seating rim. The capsule has an inner diameter at the open end and has an outer diameter. A primary element, such as a section of cylindrical wire, such as formed of metal such as stainless steel, copper, nickel, tungsten, etc., having a predetermined diameter and predetermined length, has a flat end. The primary element is sized to be retained in the cavity of the capsule. A radioactive part, such as a thin section of radioactive foil, is located on the flat end of the primary element. The flat end of the primary element can also be electroplated with radioactive material. The radioactive material can be a radioisotope, such as ¹⁰⁹Cd, ⁵⁵Fe, ²⁴¹Am, ⁵⁷Co, and ¹³³Ba, depending on the intended uses of the radiation source device. The amount and type of radioactive material is to be selected based on the particular needs of the radiation source material. A radiolucent window portion, such as formed by beryllium, is provided. The window portion has a flat front face and has a generally cylindrical sleeve portion extending rearwardly from the flat front face to define a window portion cavity. The cylindrical sleeve portion has an outer peripheral surface and an inner peripheral surface. Behind the flat front face the window also preferably provides a flat rear surface. The window portion preferably has a perimeter rim portion that has a surface that is adapted to seat against the seating rim of the open end of the capsule when the window portion is inserted into the open end of the capsule. A secondary seal is formed in the vicinity of the contact area between the surface of the perimeter rim portion and the seating rim of the open end of the capsule. If desired, adhesive can be applied between the outside surface of the sleeve portion and the inner surface of the walls of the capsule to further retain the window portion with the capsule.

The cylindrical sleeve portion is sized to be received in the open end of the capsule. The cylindrical sleeve portion has an outer diameter than is sized to tightly engage with the inside wall of the cavity of the capsule, and an inner diameter that is sized to permit the primary element to fit in the window portion cavity such that the radioactive flat end of the primary element will seat adjacent to the flat rear surface of the window portion. A primary seal, such as formed by an adhesive, e.g., an epoxy resin adhesive, may be used to retain the primary element together with the window portion. Normally, assembly can take place in a negative pressure glove box, where, for example, the primarily element with its radioactive end is adhered with its radioactive end against the flat rear surface of the window portion. Thereafter, the window portion with its attached primary element is inserted, for example, by press fitting the sleeve portion into the open end of the capsule. As noted above, it is possible to also use an adhesive to further adhere the window portion to the capsule with the radioactive primary element contained therewithin.

The invention is now briefly described below with reference to the drawings.

### BRIEF DESCRIPTION OF THE DRAWINGS

FIG. 1 is a front isometric view of a prior art radiation source device.

FIG. 2 is a front view of the prior art radiation source device of FIG. 1.

FIG. 3 is a cross-sectional view of the prior art radiation source device of FIG. 2 through view lines 3-3.

FIG. 4 is a front isometric view of an exemplary embodiment of a radiation source device of the invention.

FIG. 5 is a cross-sectional view of the exemplary embodiment of the assembled radiation source device of FIG. 4.

FIG. 6 is an exploded view of the exemplary embodiment of a radiation source device of FIG. 4.

### DETAILED DESCRIPTION OF THE INVENTION

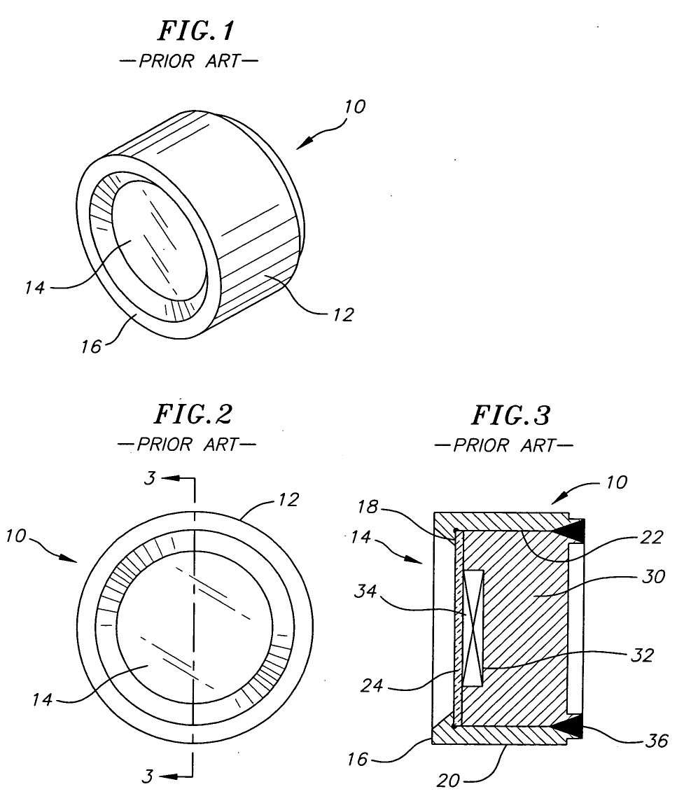

FIG. 1 is a front isometric view of a prior art radiation source device 10, shown as a generally cylindrical device. A front view of the radiation source device 10 is shown in FIG. 2 and a cross-sectional view along view lines 3-3 of FIG. 2 is shown in FIG. 3. The radiation source device 10 has a capsule portion 12 with an open front 14. The capsule is preferably made of a strong and radiopaque material, such as stainless steel, nickel-copper alloys, such as Monel®, etc. The open front 14 has an outer rim 16 with an inner seating rim 18. The capsule has an outer cylindrical surface 20 and an inner cylindrical surface 22 that defines a generally cylindrical space therein. The inner seating rim 18 projects inwardly of the inner cylindrical surface 22. A section of radiolucent material, such as a section of beryllium is used to form a radiolucent window 24, which is sized to tightly fit behind the inner seating rim 18. The beryllium window is permanently affixed in place, e.g., by brazing. A generally cylindrical plug 30 is provided that has an outer diameter that is sized to fit within the space of the capsule 12 snuggly against the inner cylindrical surface 22. The plug 30 is preferably made of a strong and radiopaque material such as stainless steel, nickel copper alloys and the like, and has a recess 32 that is sized to receive a radioactive element 34. The plug 30 with its carried radioactive element 34 is inserted into the capsule with the radioactive element 34 seated against the inside of the radiolucent window 24, so that radiation emanates from the radiolucent window 24, but not from other directions of the radiation source device 10. The plug 30 is preferably permanently affixed to the capsule 12, e.g., by fusion welding 36.

It is desirable to provide a radiation source device a small size format, and accordingly, it is desirable to be able to size radiation source devices as small as possible. For example, prior art devices have been sized to be relatively small, such as having a diameter of about 8 mm, with a window size of about 5 mm. However, properly brazing the beryllium window 24 to the capsule 22 in these small size formats becomes difficult and there can be a high defect rate. Accordingly, the design of prior art radiation source devices is not ideal.

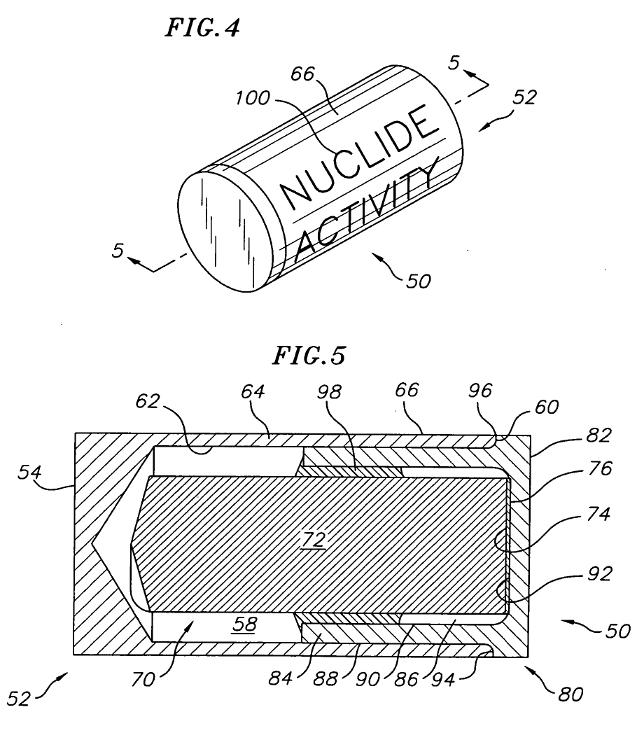

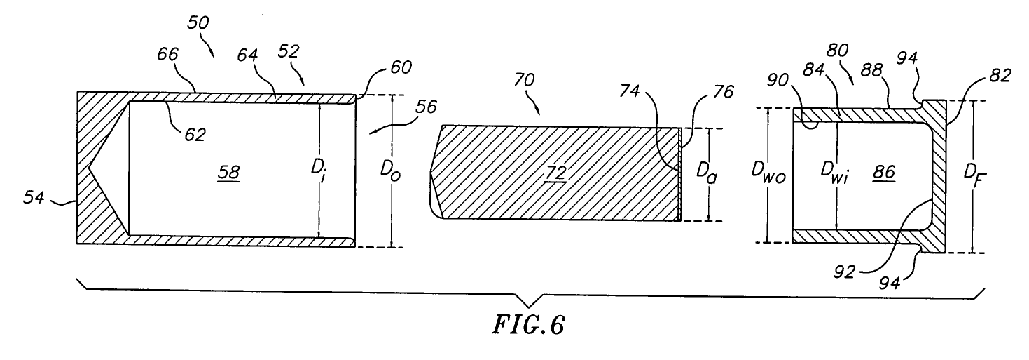

FIG. 4 is a front isometric view of an exemplary embodiment of a radiation source device 50 of the invention, FIG. 5 is a cross-sectional view of the assembled radiation source device of FIG. 4 along view lines 5-5 of FIG. 4, and FIG. 6 is an exploded view of the radiation source device 50 of FIG. 4. The radiation source device 50 comprises a capsule 52 made of a radiopaque material, such as stainless steel, nickel copper alloys and the like. The capsule 52 can have a generally cylindrical shape with a closed end 54 and an open end 56 defining a cavity 58 therein. The open end 56 has a seating rim 60. The capsule 52 has an inner diameter “Dᵢ” at the open end and has an outer diameter “Dₒ”, with the inside surface 62 of a cylindrical wall portion 64 defining the inner diameter “Dᵢ” and an outer surface 66 defining the outer diameter “Dₒ”. A primary element 70, for example as a section of cylindrical wire 72 formed of a metal such as stainless steel, copper, nickel, silver, etc., or other suitable materials, such as porous ceramic, porous glass, and ion exchange resin beads, and has a predetermined diameter Dₐ and predetermined length, and has a flat front end 74. A radioactive part 76, such as a thin section of radioactive foil, is located on the flat front end 74 of the primary element 70. The flat front end 74 of the primary element can also be electroplated with radioactive material 76. Regardless of how the radioactivity is located at the front of the element, the radioactive element can comprise a radioactive isotope, such as the following: ¹⁰⁹Cd, ⁵⁵Fe, ²⁴¹Am, ⁵⁷Co, and ¹³³Ba. The primary element 70 is sized to be fit in the cavity 54 of the capsule 52 with its radioactive end 76 facing outwardly towards the open end 56 of the capsule 52. As can be seen, the diameter “Dₐ” of the active element 70 is smaller than the inner diameter Dᵢ of the space 58 of the capsule 52. The amount and type of radioactive material is to be selected based on the particular needs of the radiation source material. A radiolucent window portion 80, such as formed by beryllium, is provided. The radiolucent window portion 80 preferably has a flat front face 82 and has a generally cylindrical sleeve portion 84 extending rearwardly from the flat front face 82 to define a window portion cavity 86. The cylindrical sleeve portion 84 has an outer peripheral surface 88 and an inner peripheral surface 90. Behind the flat front face 82, the window portion 80 also preferably provides a generally flat rear surface 92. The window portion 80 preferably has a perimeter rim portion 94 with a surface that is adapted to seat against the seating rim 60 of the open end 56 of the capsule 52 when the window portion 80 is inserted into the open end 56 of the capsule 52. A primary seal 96 is formed in the vicinity of the contact area between the surface of the perimeter rim portion 94 and the seating rim 60 of the open end 56 of the capsule 52. This secondary seal 98 can be formed by adhesive and/or welding. Also, if desired, adhesive can be applied between the outside surface 88 of the sleeve portion 84 and the inner surface 62 of the walls 64 of the capsule 52 to further retain the window portion 70 together with the capsule 52. The cylindrical sleeve portion 84 is sized to be received in the open end 56 of the capsule 52. The outer diameter D_{wo} of 5 the cylindrical sleeve portion 84 is sized to fit within the inner diameter Dᵢ of the inside wall 62 of the cavity 58 of the capsule 52, and the cylindrical sleeve portion 84 has an inner diameter D_{wi} that is sized to permit the primary element 70 to fit in the window portion cavity 86 such that the radioactive flat end 76 of the primary element 70 will seat adjacent to the flat rear surface 92 of the window portion 80. A primary seal 96, such as formed by an adhesive, e.g., an epoxy resin adhesive, is preferably used to retain the primary element 70 together with the window portion 80. For purposes of meeting governmental regulations, the outer surface 66 of the capsule 52 can bear marking 100 (e.g., “NUCLIDE ACTIVITY”), such as by engraving, to identify the radioactive source device 50 as being radioactive.

Assembly of the radiation source device 50 can take place in a negative pressure glove box, where, for example, the primary element 70 is inserted into the window portion 80 with its radioactive end 76 being seated against the inside surface 92 of the window, and with adhesive used to form the primary seal 98 between the primary element 70 and the window portion 80 to retain these portions together. Thereafter, the primary element 70 and the window portion 80 unit are fitted into the open end 56 of the capsule 52. As noted above, a snug fit will be formed between the outside surface 88 of the window portion 80 adhered with its radioactive end 76 against the flat rear surface 92 of the window portion 80. As noted above, an adhesive (such as epoxy resin) can be used to adhere the window portion 80 to the capsule 50 with the radioactive primary element 70 contained therewithin. Lastly, at the location of the secondary seal 96, further bonding may be effected, such as by adhesive and/or by welding. While welding (fusion welding, laser welding, etc.) can be used, in order to eliminate any beryllium fumes, assembly without the use of welding is desirable, and adhesives are preferable. Indeed, since welding can be eliminated, very small sized radioactive source devices can be made. For example, sources with windows as thin as 0.25 mm (or thinner) and having an diameter of about 3 mm and length of 6 mm or so can readily made with high yields and very low defect rates.

While the exemplary radioactive source device 50 of the invention is shown as have a generally elongate cylindrical shape, radioactive source device of the invention can have other shapes if desired. For example, rather than being cylindrical, the radioactive source device can be frustoconical in shape, can have a polygonal cross-section, etc.

Although embodiments of the present invention have been described in detail hereinabove in connection with certain exemplary embodiments, it should be understood that the invention is not limited to the disclosed exemplary embodiments, but, on the contrary is intended to cover various modifications and/or equivalent arrangements included within the spirit and scope of the present invention.

What is claimed is:

1. A radiation source device, comprising:
a radiolucent window portion with a front window and a sleeve portion having an outer surface and a window portion cavity therein;
a primary element with a radioactive end, which primary element is received in the window portion cavity with its radioactive end adjacent to the front window;
a capsule with an open end that is sized to receive the sleeve portion of the radiolucent window portion and the primary element.

2. The radiation source device of claim 1, wherein an adhesive is used to form a primary seal between the primary element and the radiolucent window portion.

3. The radiation source device of claim 1, wherein the capsule comprises generally cylindrical side walls and a closed end opposite the open end, wherein the open end has a seating rim, and wherein the radiolucent window portion has a perimeter rim portion that is adapted to closely engage with the seating rim.

4. The radiation source device of claim 3, wherein a secondary seal is formed in the vicinity of where the perimeter rim portion of the radiolucent window portion contacts with the seating rim of the capsule.

5. The radiation source device of claim 4, wherein the radiolucent window portion and the capsule are attached together by at least one of welding, adhesive and compression fitting.

6. The radiation source device of claim 1, wherein the radiolucent window portion is formed of beryllium.

7. The radiation source device of claim 1, wherein the a primary element comprises one of metal, porous glass, porous ceramic and ion exchange resin beads, and wherein the radioactive end is formed by electroplating or attaching a radioactive layer to the front of the primary element.

8. The radiation source device of claim 1, wherein the radioactive end comprises a radioisotope selected from one of ¹⁰⁹Cd, ⁵⁵Fe, ²⁴¹Am, ⁵⁷Co, and ¹³³Ba.

9. The radiation source device of claim 1, wherein the capsule comprises radiopaque material.

10. A radiation source device, comprising:
a radiolucent window portion with a front window, a sleeve portion having an outer surface and an inner surface, and a window portion cavity therein;
a primary element with a radioactive end and cylindrical walls, which primary element is received in the window portion cavity with its radioactive end adjacent to the front window, wherein a primary seal is formed between the cylindrical walls of the primary element and the inner surface of the sleeve portion of the radiolucent window portion;
a capsule with an open end that is sized to receive the sleeve portion of the radiolucent window portion and the primary element.

11. The radiation source device of claim 10, wherein the primary seal comprises adhesive.

12. The radiation source device of claim 10, wherein the capsule comprises generally cylindrical side walls and a closed end opposite the open end, wherein the open end has a seating rim, and wherein the radiolucent window portion has a perimeter rim portion that is adapted to closely engage with the seating rim, wherein a secondary seal is formed in the vicinity of where the perimeter rim portion of the radiolucent window portion contacts with the seating rim of the capsule.

13. The radiation source device of claim 12, wherein the radiolucent window portion and the capsule are attached together by at least one of welding, adhesive and compression fitting.

14. The radiation source device of claim 10, wherein the radiolucent window portion is formed of beryllium.

15. The radiation source device of claim 10, wherein the a primary element comprises one of metal, porous glass, porous ceramic and ion exchange resin beads, and wherein the radioactive end is formed by electroplating or attaching a radioactive layer to the front of the primary element.

16. The radiation source device of claim 10, wherein the radioactive end comprises a radioisotope selected from one of ¹⁰⁹Cd, ⁵⁵Fe, ²⁴¹Am, ⁵⁷Co, and ¹³³Ba.

17. The radiation source device of claim 10, wherein the capsule comprises radiopaque material.
